(19) United States
(12) Patent Application Publication
(10) Pub. No.: US 2009/0216200 A1
(43) Pub. Date: Aug. 27, 2009

(54) System for Introducing an Agent Into a Blood Vessel

(76) Inventors: John S. Geis, Bad Zwischenhahn (DE); Michael Braun, Backnang (DE)

Correspondence Address: Mark J. Pandiscio;Pandiscio & Pandiscio, 470 Totten Pond Road, Waltham, MA, 02451-1914, US

(21) Appl. No.: 11/885,976

(22) PCT Filed: Mar. 13, 2006
(86) PCT No.: PCT/DE06/00435

§ 371 Date: Aug. 29, 2008

(30) Foreign Application Priority Data
Mar. 14, 2005 (DE) 10 2005 011 656.6

(51) Int. Cl. A61M 25/14

(52) U.S. Cl. 604/246

(57) ABSTRACT

The present invention relates to a system for introduction of an agent into a blood vessel (G), wherein the system (1) comprises a wire (10) having a proximal end (114, 121) and a distal end (112) as well as lumen (115) extending between these ends, wherein the system is characterized in that a matrix (13) for release of the agent is provided within the lumen (115), wherein the matrix (13) extends over at least part of the length of the wire (10). Thereby an oxygenation system for cerebral vessels can be provided.

The present invention relates to a system for introducing an agent into a blood vessel and in particular to a cerebral oxygenation system.

Infarcts are incidences, wherein due to an undersupply of oxygen caused by insufficient blood flow a tissue death, a so called necrosis, occurs. In most cases an infarct occurs due to partial or complete occlusion of vessels. These vascular occlusions can be caused by blood clots, which are also referred to as thrombi, or by other material (lime, tissue particles or fat depositions) which is washed in with the blood.

Due to the hindered or interrupted blood flow to the tissue and further vessels which in the direction of the blood flow are located behind the vascular occlusion, cells and entire tissue parts quickly necrose and a failure of the respective functions occurs. With a cardiac infarction or myocardial infarct, for example the cardiac muscle is destroyed due to lack of oxygen and the thus caused dysmetabolism of the cells. With a cerebrovascular accident or apoplexy, the cerebral nerve cells and the cell tissue rapidly suffer and die. Also in this case an undersupply and loss of functionality of parts of the brain is caused by a lack of oxygen and the thus resulting dysmetabolism.

In order to treat the undersupplied area behind the occlusion, which is also referred to as the ischemic area, the cells in this area have to be provided with a large amount of oxygen. Up to now a systemic treatment is being used. Herein, the oxygen may be administered to the patient for example over a mask. Even though the inspiration results in a 100% uptake of the oxygen in the blood, due to the existing occlusion, the required amount of oxygen will, however, not be guided to the cells of the blood vessel in the ischemic area. Locally, the saturation with oxygen, which would be necessary to avoid necrosis of the cells, is not sufficiently achieved.

The problem to be solved by the present invention is thus to provide a system which allows for rapidly providing areas of a blood vessel, in particular an ischemic area, with an agent, in particular with an agent for providing the required amount of oxygen.

The invention is based on the finding that this problem can be solved by using a system wherein a matrix for delivery of the agent is arranged in the interior of a rigid introduction system.

In particular, the problem is solved by a system for introducing an agent into a blood vessel, wherein the system comprises a wire having a proximal end and a distal end as well as a lumen extending between these ends. The system is characterized in that a matrix for release of the agent is provided in the lumen, wherein the matrix extends at least over a part of the length of the wire.

With the system according to the invention, the introduction of the agent is preferably effected into the blood stream. In particular, the introduction is effected into a partially or completely occluded ischemic area of a blood vessel. The agent which is to be delivered into this area can be received within the matrix which is provided according to the invention and can be released from the matrix by different release mechanisms. In particular, the agent can be released into the ischemic area by local diffusion, by pressure and/or by temperature change. The matrix thus functions as a substrate and/or carrier for the agent which is to be introduced into the blood vessel.

By combining a wire with a release matrix a synergetic effect can be achieved. On the one hand, the utilization of a wire as holder for the release matrix provides a rigid arrangement. The wire is preferably made of metal or a metal alloy. It is particularly preferred to use nickel-titanium-alloys, such as nitinol, stainless steel and/or platinum as material. Due to the stability, a vascular occlusion can be penetrated with the inventive system. Thereby, a passage to the ischemic area of the blood vessel is created. Such a penetration can not be realized with a flexible system such as a plastic tube. In addition, the utilization of a wire allows for providing the stability even with a wire having a small diameter. Such a small diameter is of particular importance for the treatment of apoplexy, as the vascular occlusion therein occurs in vessels having a very small inner diameter. The cerebral vessels can have a diameter of 1.5 mm or less.

On the other hand, the agent, which is to be introduced into the area of the blood vessel, is held in the enclosed release matrix. By arranging the matrix in the interior of the wire, the outer diameter of the system is not affected thereby and the matrix is not damaged during the penetration through a vascular occlusion. In addition, the provision of a matrix for the release of the agent influences the quality and quantity of the agent which is released there through. In contrast to the introduction of an agent into a blood vessel via a lumen of a wire or catheter without a matrix, the release can be controlled or dosed, respectively, via the matrix. In particular, with a liquid agent a dripping or spraying of the agent out of the lumen can be avoided. Also the quality of the release of the agent is increased by providing a matrix. In particular, a liquid agent can be atomized by the matrix, that means the drop size of the released agent is minimized. The drop size can for example vary from 1 μm to 300 μm. The matrix is preferably arranged in the lumen of the wire such that the entire inner diameter of the wire is filled.

Furthermore, the combination of a wire and an enclosed matrix also provides advantages over systems, wherein a catheter, though which the agent is to be delivered, is guided over a guide wire. With this system the guide wire may serve for penetration of the vascular occlusion. The introduction of the agent, however, has to be effected via the lumen of the catheter. Herein, the agent can only reach the ischemic area via the passage which was created in the occlusion by the penetration of the occlusion with the guide wire. The introduction of the agent in a sufficient manner with therapeutic effect is almost impossible. Furthermore, the time necessary for guiding the catheter over the guide wire represents a loss of time, which can be in particular crucial with the treatment of apoplexy. With the system according to the invention, in contrast, a release of the agent is possible as soon as the wire has been guided into the ischemic area.

In the system according to the invention, the matrix is preferably positioned in the section of the distal end of the wire. The distal end of the wire, in particular the distal opening, which represents the distal end of the lumen, is the location at which the lumen is in contact with the ischemic area. According to the invention, the enclosed matrix is brought into the lumen to a certain extent in this section and can be attached to the wire, e.g. adhered to it. Thereby, on the one hand the entry of blood into the lumen is essentially hindered and on the other hand an uncontrolled release of the agent from the lumen into the ischemic area is prevented.

According to the invention, it is possible to only provide the matrix in the distal section of the wire. Thereby, less material is necessary for manufacturing the matrix. The remaining free area of the lumen can be filled with the liquid agent, which is to be released. Hence, a large amount of agent can be received in the lumen and can be available for release. Only in the distal section, where the release takes place, the matrix works for optimizing the quality and quantity of the released agent.

According to the invention, it is, however, also possible to provide the matrix over the entire length of the wire, that means from the distal end to the proximal end and potentially also beyond these ends.

This embodiment has the advantage that the matrix can serve as a guiding means for the agent over the entire length of the wire. According to the wick principle, a continuous release of the agent by diffusion at the distal end of the wire can be assured by diffusion within the matrix.

According to one embodiment, the wire has a release region within its distal section, where at least one release opening is provided in the wall of the wire. This at least one release opening extends between the inner diameter and the outer diameter of the wire. Additional release locations are provided by these openings in addition to the distal opening of the lumen and the available release area can thus be increased. The openings may for example be round holes, which have been formed in the wall of the wire by laser treatment.

It is, however, also possible to provide the at least one opening as a spiral opening in the wall of the wire. Thereby, the distal section of the wire receives the shape of a spiral. With a spiral the release area is larger compared to the release area provided by holes. In addition, the distal section of the wire is provided with increased flexibility. Thereby, the introduction of the wire into a blood vessel can be facilitated and the risk of injury for the patient can be decreased.

The length of the release region, wherein the at least one release opening is provided, is small in relation to the length of the entire wire. Depending on the application, the length of the wire can be in the range of 30 mm to 3000 mm, while the release region may preferably have a length of 10 mm to 100 mm.

When using a helical-shaped opening as a release opening, the release region can be integral with further regions of the wire. For manufacturing the inventive wire, it is, however, also possible to manufacture the spiral separately and to attach the spiral to a hollow wire, that means to attach it to the distal end thereof. Thereby, the manufacturing of the wire is facilitated. In addition, it is advantageous, as the spiral can consist of a different material than the hollow wire, if the spiral is manufactured separately. The spiral may for example be made of platinum while the hollow wire may be made of nitinol or stainless steel. In such a system the platinum spiral may serve as a marker, which allows for a better navigation for the surgeon. A separate marker is not necessary in this case. In addition, the system can be produced at low costs, as the long hollow wire can be made of a comparatively inexpensive material. The connection of the spiral with the hollow wire for forming the inventive wire can be effected by welding or soldering.

In this embodiment it is particularly preferable, if the hollow wire has a bar at its distal end, which extends from the distal end of the lumen to the distal end of the spiral. The distal end of the spiral may be attached to the distal end of the bar. Additionally, the spiral may be fixated and/or attached at the distal end of the hollow wire or in the distal section of the hollow wire.

Furthermore, it is possible to realize the release region by a tube, in particular a plastic tube, e.g. a PTFE tube, with holes introduced into the walls thereof and which is put onto the distal end of the hollow wire and is connected therewith. In this embodiment, the release region is formed by the part of the plastic tube which extends beyond the hollow wire and where holes are provided in the tube walls. The tube may be attached to the outside of the hollow wire in the distal section for example by adhering.

In the embodiment, where the release region is designed as a spiral or where holes are provided in the release region, the matrix is preferably arranged such that it completely covers the holes and/or the spiral opening. Thereby, a maximal release area can be provided. For the positioning of the matrix within the lumen of the wire, it is, however, only essential that the matrix covers the transition to the release region in order to avoid escaping of the agent from the lumen where no matrix structure is present.

A porous material, in particular a porous plastic is preferably used as the material of the matrix. The agent can be received in the pores. It is in particular preferable to use polyfluoroethylene (PTFE), in particular expanded polyflouroethylene (EPTFE). The pore size within the matrix, according to the invention, may be within the range of 1 μm to 200 μm. Such a matrix allows for achieving a continuous release of the agent.

The matrix may be inserted by foaming into the lumen of the wire. Preferably, the matrix is, however, prefabricated and is a thread/monofilament, which is guided into the lumen.

The agent, which can be released with the system according to the invention, is preferably a liquid agent. Particularly preferably for example a liquid oxygen carrier and/or a liquid medicament can be released with the inventive system. A preferred example of an oxygen carrier is a perfluorocarbon solution, which is saturated with oxygen. Perfluorocarbons are suitable for the treatment of ischemic areas in particular due to their low surface tension and their high solubility for oxygen.

According to one embodiment, the outer diameter of the wire may decrease over a region between the proximal and the distal end. It is particularly preferable to only shape the distal end of the wire conically. The conical shape may be created by partially grinding the outside of the wire. Due to the tapering at the tip of the wire, the introduction of the system into the blood vessel can be facilitated.

If the wire is a hollow wire with a spiral attached to it, the tapering is preferably provided at the distal area of the hollow wire. The outer diameter of the spiral in contrast is constant. Due to the tapering of the distal end of the hollow wire, a spiral having a smaller diameter can be used and can easily be fixated to the hollow wire, for example by sliding it onto the hollow wire. Particularly preferably, the outer diameter of the spiral is chosen to correspond to the outer diameter of the hollow wire at its cylindrical part. The inner diameter of the spiral, in contrast, may be chosen to be slightly larger than the diameter of the lumen of the hollow wire. Thereby, the diameter of the matrix which extends into the spiral area may expand from the diameter of the lumen to the inner diameter of the spiral in the transition area between the hollow wire and the spiral.

In this embodiment, the spiral is slid onto the conical part of the wire until the proximal end of the spiral abuts with the distal end of the cylindrical part of the hollow wire. The spiral can be connected or attached to the hollow wire at that location. The wire thus obtains an outer diameter, which remains constant over its overall length. In this embodiment, the release region is formed by the part of the spiral which extends beyond the distal end of the hollow wire.

The hollow wire may comprise a bar at its distal end where the distal opening of the lumen is located for additional fixation of the spiral or of a plastic tube to the hollow wire. The bar can be created by removing, for example grinding of, a part of the lateral surface of the hollow wire at its distal end, and is thus an integral component of the hollow wire. The distal end of the bar can serve as an additional attachment point for a spiral or a plastic tube. At that location, a spiral may for example be fused with the bar. Thereby, the risk of injuring a patient is minimized and the distal end may therefore also be referred to as an atraumatic wire head.

The bar ensures an attachment of the spiral or the tube, which prevents detaching of these components from the hollow wire during introduction or removal from the blood vessel.

The inventive system may comprise a connecting device, which can detachably be attached to the proximal end of the wire and may serve for introduction of the agent into the lumen. This connecting device particularly comprises a valve, through which the proximal end of the wire can be introduced into a closed container, where the agent to be introduced is present.

Furthermore, the connecting device may additionally or alternatively have an inlet for a syringe, a so called side port. This inlet preferably terminates within the connecting device behind the valve, that means in the main channel of the valve. Thereby, pressure can be generated in the lumen of the wire by means of a syringe.

The fact that the connecting device is only temporarily connected with the wire of the system, has several advantages. Firstly, the wire after having been detached from the connecting device may be available for further applications. The wire of the system may for example be used as a guide wire for a micro catheter. Agents for dissolving thrombi may be guided to a location before the occlusion in the blood vessel via this catheter. Also while the catheter is being introduced, the release matrix may continue to release the agent behind the occlusion in the blood vessel. For this purpose, the wire of the system according to the invention may be closed with an end piece at the proximal end after the connecting device has been removed. This end piece, which may be plug or a lid, prevents agent from coming out at the proximal end of the wire and additionally serves for maintaining pressure conditions within the lumen of the wire.

It is also possible to provide receiving apertures in the wire wall in the section of the proximal end of the wire. Thereby, the receiving area for the agent to be introduced can be increased. Also these receiving apertures may for example be introduced into the wire wall by laser treatment. In this case, an end piece, which surrounds the proximal section, where the apertures are provided, will be used for closing the proximal end.

The invention will hereinafter be described with reference to the attached drawings, which relate to possible embodiments of the invention, wherein:

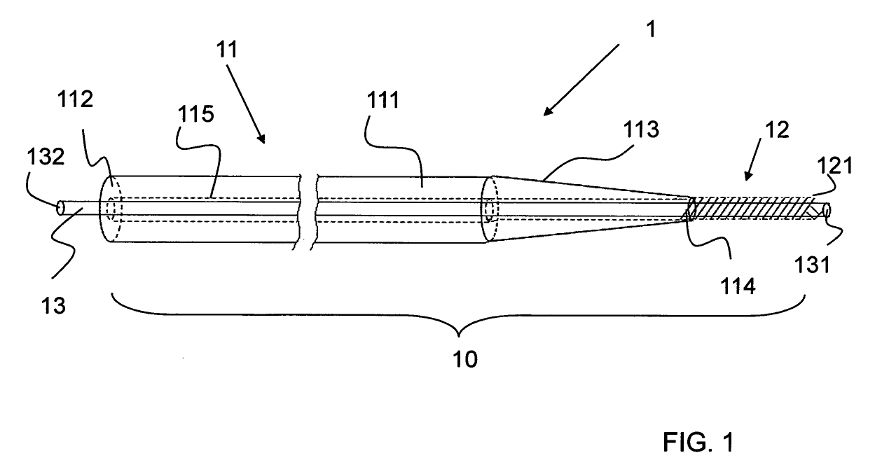

FIG. 1: shows a schematic depiction of a first embodiment of the system according to the invention;

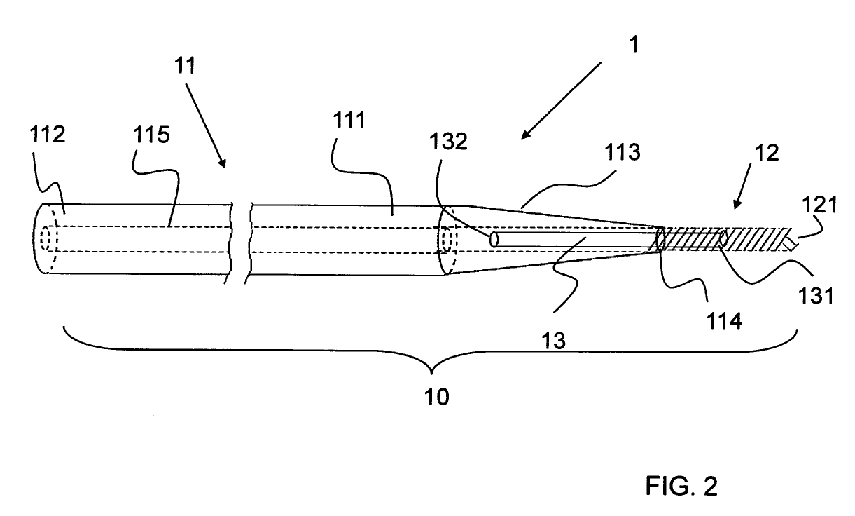

FIG. 2: shows a schematic depiction of a second embodiment of the system according to the invention;

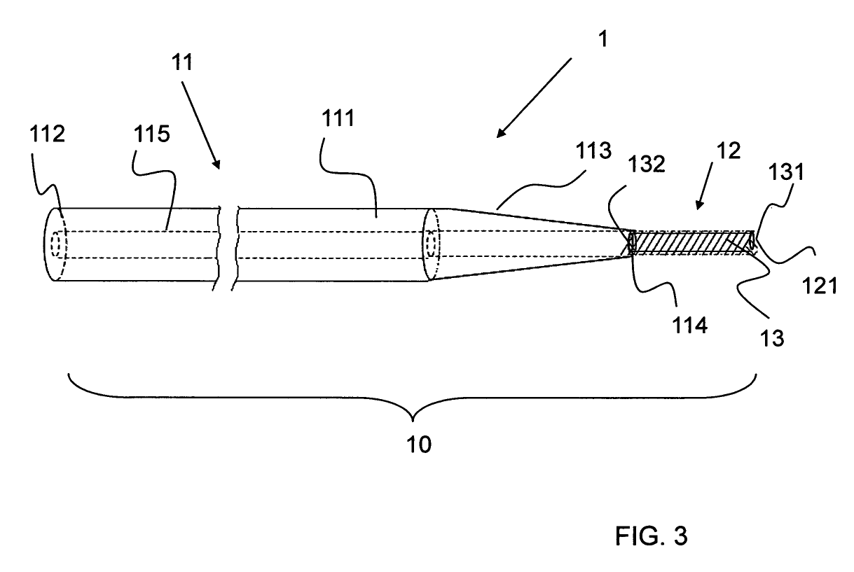

FIG. 3: shows a schematic depiction of a third embodiment of the system according to the invention;

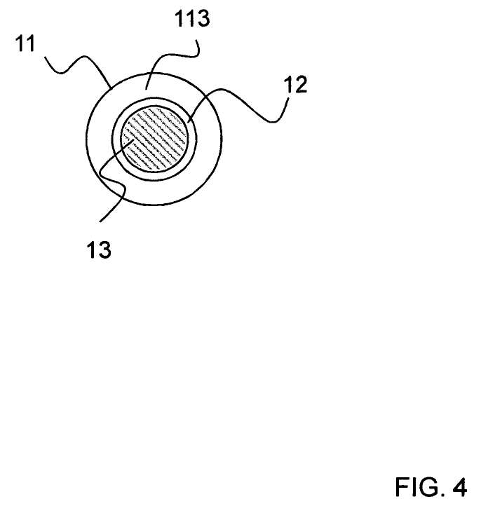

FIG. 4: shows a schematic front view of an embodiment of the system according to the invention;

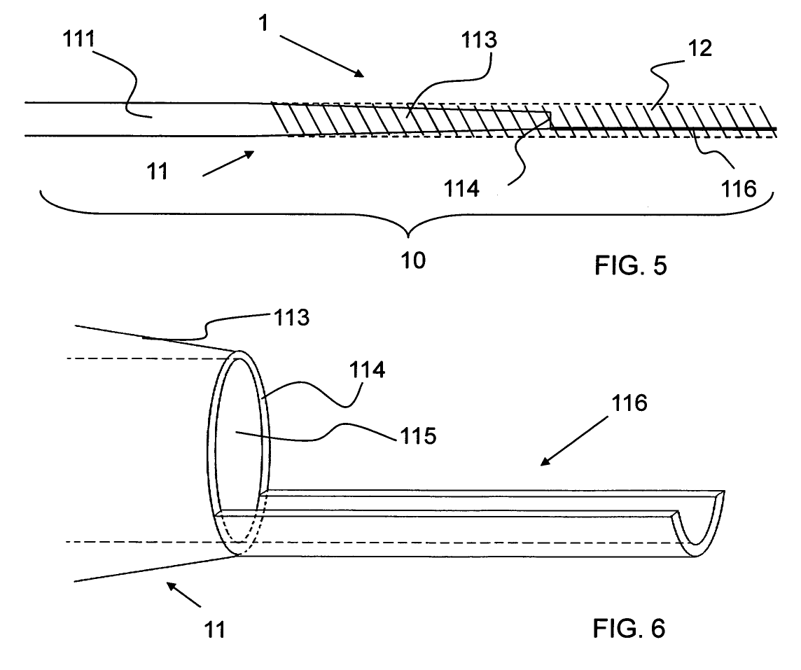

FIG. 5: shows a schematic detail view of the release region of a preferred embodiment of the system according to the invention;

FIG. 6: shows a schematic detail view of the distal end of the hollow wire according to the embodiment of the inventive system according to FIG. 5;

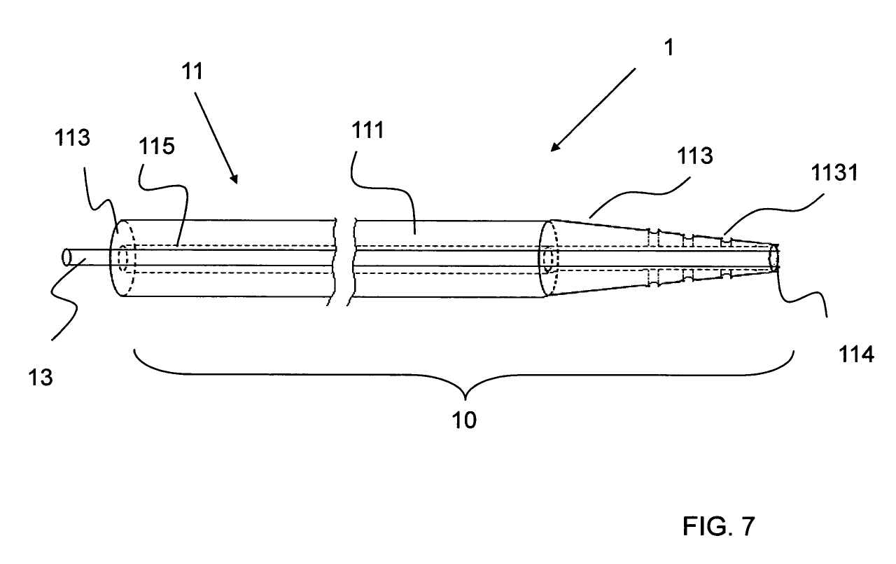

FIG. 7: shows a schematic depiction of a forth embodiment of the system according to the invention;

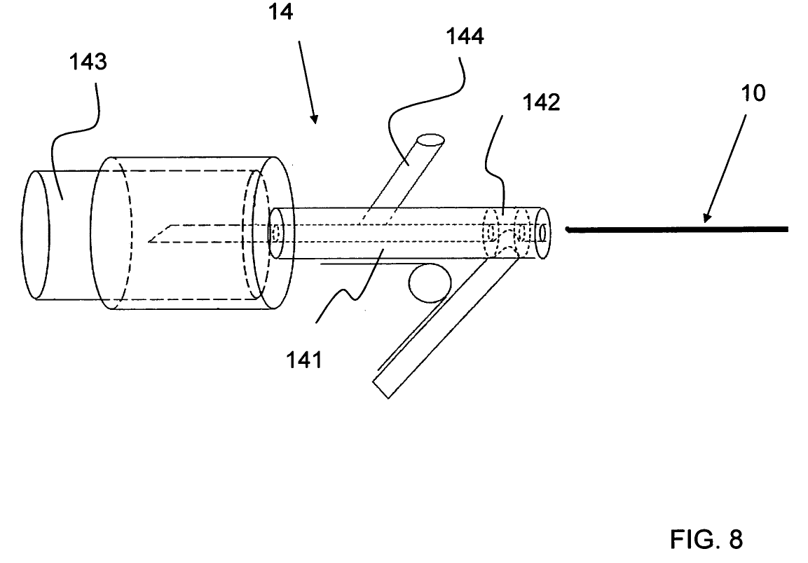

FIG. 8: shows a schematic depiction of an embodiment of a connecting device of the introduction system according to the invention;

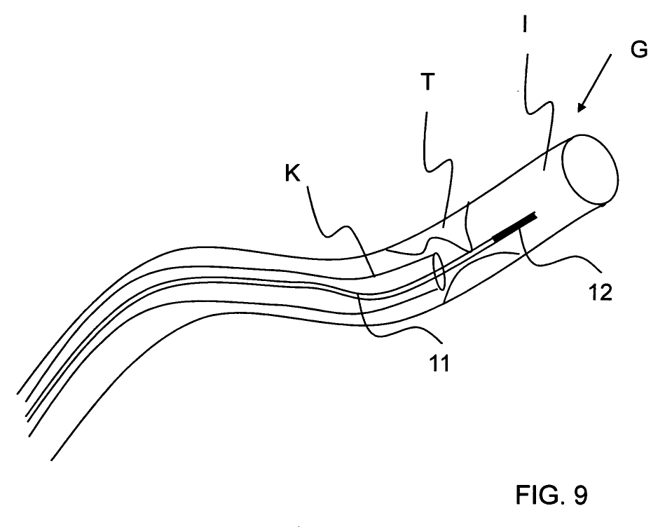

FIG. 9: shows a schematic depiction of the system according to the invention in a blood vessel; and

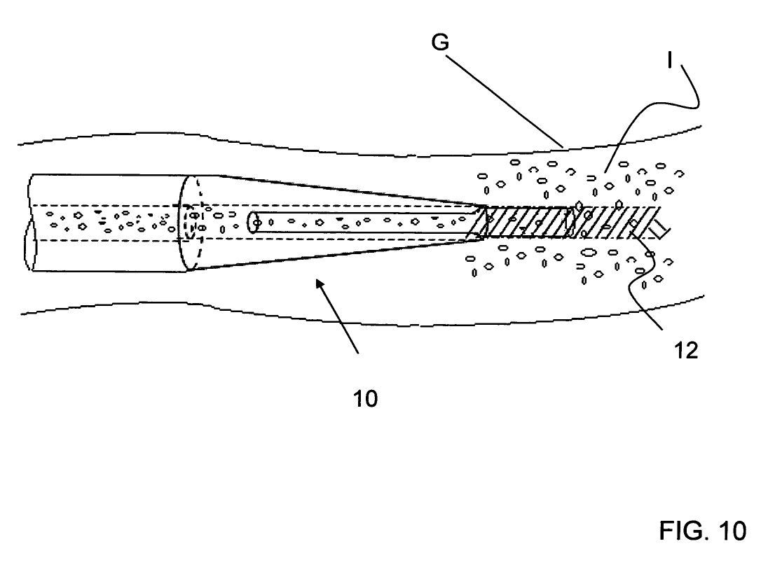

FIG. 10: shows a schematic detail view of the release region of a system according to the invention in a blood vessel.

The depictions are not to scale.

In FIG. 1 an embodiment of the introduction system 1 according to the invention is schematically shown. In this embodiment the system 1 comprises a wire 10. This wire 10 consists of a hollow wire 11 and a spiral 12.

The hollow wire 11 has a cylindrical part 111 which extends from the proximal end 112 of the hollow wire 11. This part 111 is only shown partially. In one embodiment the cylindrical part 111 extends for example over 1500 mm. A conical part 113, wherein the outer diameter of the hollow wire 11 decreases, is adjacent to the cylindrical part 111 of the hollow wire 11. The hollow wire 111 thus has a smaller outer diameter at the distal tip 114. A lumen 115 extends within the hollow wire 11 over the cylindrical part 111 and the conical part 113. The diameter of the lumen 115, that means the inner diameter of the hollow wire 11, is constant over the length of the hollow wire 11.

In the depicted embodiment, a spiral 12 is adjacent to the distal tip 114 of the hollow wire 11, wherein the spiral 12 is attached to the tip 114. The inner diameter of the spiral 12 herein represents an extension of the lumen 115 of the hollow wire 11 and preferably has the same diameter as the lumen 115.

In the depicted embodiment, a matrix 13 extends through the lumen 115 of the hollow wire 11 and the spiral 12. In the depicted embodiment, the matrix 13 consists of a thread or monofilament made of ePTFE and serves for releasing an agent, which is guided there through or which is contained therein, respectively. As can be derived from FIG. 4, the matrix 13 essentially fills the entire cross section of the lumen 115 of the hollow wire 11 and the spiral 12, respectively. In the area of the spiral 12, the matrix 13 comes in contact with the surroundings at the clearances between the spiral coils. In addition, the distal end 131 of the matrix 13 is in contact with the surroundings at the distal end 121 of the spiral 12.

In the depicted embodiment, the matrix 13 extends beyond the hollow wire 11 at the proximal end 112 of the hollow wire 11. The matrix 13 may, however, also terminate flush with the end 113 of the hollow wire 11.

In FIG. 2 a second embodiment of the system 1 according to the invention is shown. The arrangement of the system 1 essentially corresponds to the arrangement of the first embodiment. The components are referred to with corresponding reference numbers and their function will not be described again.

In contrast to the first embodiment, the matrix 13 in the second embodiment only extends over the section of the spiral 12 and a part of the lumen 115 of the hollow wire 11. In the depicted embodiment, the matrix 13 terminates in a distance to the distal tip 121 of the spiral 12. It is, however, also possible according to the invention that the matrix 13 extends to the tip 121 of the spiral 12. Also the proximal end 132 of the matrix 13 is not limited to the depicted position within the conical part 113 of the hollow wire 11. The matrix 13 may also terminate in the cylindrical part 111 of the hollow wire 11. It is only essential that the matrix 13 covers the lumen 115 of the hollow wire 11 at the distal tip 114 of the hollow wire 11.

In FIG. 3 a third embodiment of the introduction system 1 of the invention is shown. The arrangement of this embodiment also essentially corresponds to the embodiment shown in FIG. 1. In the depicted embodiment, the matrix 13, however, is only provided in the area of the spiral 12. Herein, the distal end 131 of the matrix 13 may be spaced from the distal end 121 of the spiral 12, that means the matrix 12 may end within the spiral 12. The matrix 13 will, however, always be positioned such that the proximal end 132 thereof lays at least at the tip 114 of the hollow wire 11 or even within the lumen 115 of the hollow wire 11.

In FIG. 5, a preferred embodiment of the release region of the system 1 of the invention is shown. In this embodiment, the spiral 12 and the cylindrical part 111 of the hollow wire 11 have the same outer diameter. The spiral 12 is slid onto the hollow wire 11 far enough to essentially cover the entire conical part 113 of the hollow wire 11. The entire wire 10 thus obtains a continuous outer diameter. In addition, a bar 116 is provided at the distal end 114 of the hollow wire 11. The spiral 12 is connected to the bar 116 and thus to the hollow wire 11 at the distal end of the bar 116. Additionally, the spiral 12 in this embodiment may be attached at the beginning of the conical part 113, that means at the distal end of the cylindrical part 111.

This kind of connection of the spiral 12 with the hollow wire 11, which is shown in FIG. 5, is preferably also realized with the embodiments shown in FIGS. 1 to 3.

In FIG. 6 a detailed view of the distal end 114 of the hollow wire 11 with a bar 116 according to FIG. 5 is shown. From this view it can be derived that the bar 116 is a continuation of the outer wall of the hollow wire 11.

In FIG. 7 a fourth embodiment of the system 1 according to the invention is shown. Herein, the wire 10 of the system 1 only comprises a hollow wire 11. This hollow wire 11 also has a cylindrical 111 and a conical part 113. In the conical part 113 openings 1131 in the form of holes are provided in the proximity of the distal end 114 of the wire 11. The openings 1131 extend to the lumen 115 of the hollow wire 11. In the depicted embodiment, the openings 1131 are positioned perpendicularly to the axis of the hollow wire 11. According to the invention, it is, however, also possible to provide the openings 1131 at a different angle to the axis. Also the number and distribution of the openings 1131 is not limited to the depicted embodiment.

In the lumen 115 a matrix 13 is provided. In the depicted embodiment, the matrix 13 extends over the entire length of the hollow wire 11. It is, however, also possible to provide the matrix 13 only in the section, where the openings 1131 are introduced into the wall of the hollow wire 11.

In the fourth embodiment, the matrix 13 thus is in contact with the surroundings at the distal end 114 of the hollow wire 11 and via the openings 1131 in the wall of the hollow wire 11.

The usage of the system 1 of the invention and its advantages will hereinafter be described again with reference to the drawings by the example of the treatment of a apoplexy patient. For the purpose of better understanding reference is made to the first embodiment, which is shown in FIG. 1, if not specified differently.

In FIG. 9 a blood vessel, in particular a cerebral blood vessel of a stroke patient is schematically shown. The blood vessel G is at least partially occluded by thrombi T. Thereby, the area I, which in the direction of the blood flow is behind the occlusion T, becomes ischemic.

In this case, a patient may now be punctuated at the groin, in particular in the area of the brachial artery or the femoral artery. The wire 10 of the system 1 can be introduced via this punctuation and can be guided to the cerebral vessels G. The spiral 12 provided on the hollow wire 11 may herein serve as a marker for the surgeon, so that he can easily monitor the progression of the wire 10. The wire 10 will be pushed forward up to the occlusion T which is formed by the thrombi and will be forced there through. Due to the penetration through the occlusion T, the section of the spiral 12 reaches the ischemic area I. In the depicted position in FIG. 9 the wire 10 is pushed so far into the ischemic area I that the entire spiral 12 is present there.

Thereby, when using the system 1 according to the invention, an oxygenation of the vessels behind an occlusion may already occur while the occlusion T still exists.

The release of the agent out of the system according to the invention is indicated in FIG. 10. The agent is represented by the dots.

The filling of the system 1 with the agent to be released for the oxygenation of the ischemic area I may be carried out with the system 1 of the invention according to the embodiment of FIG. 1 as follows. Before the wire 10 is guided in the blood vessel G to the ischemic area I, the proximal end 112 of the wire 10 is introduced into a connecting device 14.

Such a connecting device 14 is exemplarily shown in FIG. 8. The proximal end 112 of the wire 10 is introduced into a container 143 wherein the agent to be introduced is present, via a main channel 141, wherein a valve 142 is provided. This agent may for example be a liquid oxygen carrier or a medicament solution. Particularly preferably, a perfluorocarbon solution saturated with oxygen is used.

By immersing the wire 10 with its proximal end 112 into the agent, the matrix 13, which is provided in the wire 11 of the system 1, comes in contact with the agent. The agent can enter the matrix 13 and soak it. Additionally or alternatively, in particular with embodiments, where the matrix 13 does not extend to the proximal end 112 of the wire 10, pressure may be applied via an inlet 144 at the connecting device 14 for example by means of a syringe (not shown). The agent is pressed into the lumen 115 and the matrix 13 by the applied pressure.

This applied pressure ensures that the agent can be released via the matrix 13 at the distal end 114 of the hollow wire 11, that means in the area of the spiral 12. The pressure is kept low enough so that a dripping from or spraying out of the matrix 13 is prevented. Preferably, only an initial pressure is applied. Due to the release matrix 13, which is provided in the lumen 115, the agent will subsequently be sucked to the tip 114 of the hollow wire 11 and thus to the area of the spiral 12 by the wick principle. After filling or loading of the wire 10 with the agent, the connecting device 14 which is detachably connected to the wire 10, can be removed. Instead of the valve an end piece (not shown) for closing the proximal opening of the lumen 115 may then be placed on the proximal end 112 of the wire 10, whereby coming out of the agent is prevented and the established pressure conditions within the wire 10 are maintained.

The wire of the system 1 of the invention, may be used as a guide wire for a catheter K, in particular a micro catheter. The catheter can be guided over the wire 10, as soon as the wire 10 has reached the ischemic area. Thereby an oxygenation of the ischemic area I can be realized at an early point in time. The micro catheter K is moved to a location in front of the occlusion or thrombi T, respectively. An arterial thrombolysis with dissolving agents is carried out via the micro catheter K.

The invention is not limited to the depicted embodiments.

The outer diameter of the wire or hollow wire at its largest point may be in the range of 0.3 mm to 1 mm and preferably in the range of 0.3 to 0.4 mm. The diameter of the inner lumen of the wire or hollow wire may be in the range of 0.1 to 0.3 mm, preferably in the range of 0.15 to 0.25 mm and may particularly preferably be at 0.2 mm.

The spiral, which according to the invention may be provided at the distal end of the wire, is preferably a platinum spiral. It may have an outer diameter of 0.3 mm to 1.0 mm, preferably 0.4 mm. The inner diameter may be in the range of 0.1 mm to 0.3 mm, preferably at 0.25 mm.

The matrix, that means the substrate or carrier for the agent to be delivered according to the invention may preferably consist of ePTFE and have pores with a size of 1 μm to 200 μm, preferably in the size of 80 μm. The diameter of the matrix may be in the range of 0.1 to 0.3 mm, preferably in the range of 0.15 to 0.25 and particularly preferably at 0.2 mm.

According to one embodiment, the length of the system, in particular of the wire or hollow wire, respectively, is 1800 mm, wherein it has a continuous diameter over 1500 mm from the proximal end and has a conical shape at the distal end.

With the inventive system the opportunity is provided to deliver agents, such as for example oxygen carrier in an easy and rapid way into an area of a blood vessel which is limited by a vascular occlusion.

1. System for introduction of an agent into a blood vessel (G), wherein the system (1) comprises a wire (10) having a proximal end (114, 121), a distal end (112) and a lumen (115) extending between these ends, wherein a matrix (13) for release of the agent is provided within the lumen (115), and further wherein the matrix (13) extends over at least part of the length of the wire (10).

2. System according to claim 1, wherein the matrix (13) is arranged in the area of the distal end (114, 121) of the wire (10).

3. System according to claim 1, wherein the matrix (13) extends over the entire length of the wire (10).

4. System according to claim 1, wherein the outer diameter of the wire (10) decreases towards the distal end (114, 121) of the wire (10).

5. System according to claim 1, wherein the wire (10) has a release region (12, 1131) at its distal end, and further wherein in the wire wall at least one release opening (1131) is provided.

6. System according to claim 5, wherein the release area is formed by a spiral (12).

7. System according to claim 1, wherein the matrix (13) consists of a porous plastic.

8. System according to claim 1, wherein the system comprises a connecting device.

9. System according to claim 1, wherein the matrix (13) consists of ePTFE.

10. System according to claim 8, wherein the connecting device is detachably attached at the area of the proximal end (112) of the wire (10) and serves for introduction of the agent into the lumen (115).
